(19) United States
(12) Patent Application Publication
(10) Pub. No.: US 2011/0276146 A1
(43) Pub. Date: Nov. 10, 2011

(54) PROSTHETIC IMPLANT DEVICE COMPRISING A SEALING ARRANGEMENT AND A CHAMBER ADAPTED TO RECEIVE DEBRIS PARTICLES

(76) Inventors: David Segal, Jerusalem (IL); Ronny Shabbat, Kibbutz Yizre'el (IL)

(21) Appl. No.: 13/145,799

(22) PCT Filed: Feb. 11, 2010
(86) PCT No.: PCT/IL10/00120

§ 371 Date: Jul. 22, 2011

(30) Foreign Application Priority Data
Sep. 14, 2009 (GB) 0916038.3

(51) Int. Cl. A61F 2/30

(52) U.S. Cl. 623/23.4

(57) ABSTRACT

A prosthetic implant device designed to protect against debris-related dysfunction when implanted in a living body, the device including: (a) a ball-and-socket prosthesis having: a ball component having a first articulating surface, and a socket component having a second articulating surface, the socket component adapted to receive the ball component, to form an articulating region disposed directly between the surfaces, whereby the first surface is adapted to move in a relative motion with respect to the second surface; (b) a sealing arrangement, associated with the prosthesis, having a sealing member including a sealing face disposed substantially opposite, and abutting a portion of the first articulating surface, to form a sealing region adapted to obstruct a discharge of debris particles disposed directly between the articulating surfaces, from the prosthesis, via the sealing region, and (c) a chamber, associated with the prosthesis, and adapted to receive and contain the debris particles.

This application draws priority from Great Britain Patent Application No. GB0916038.3, filed Sep. 14, 2009, which draws priority from U.S. Provisional Patent Application Ser. No. 61/153,659, filed Feb. 19, 2009, and from U.S. Provisional Patent Application Ser. No. 61/175,502, filed May 5, 2009.

### FIELD AND BACKGROUND OF THE INVENTION

The present invention relates to prosthetic implants, and, more particularly, to devices for containing wear debris in prosthetic implants.

In summarizing state-of-the-art knowledge pertaining to hip arthroplasty, Huo and Cook (What's New in Hip Arthroplasty, J Bone Joint Surg Am, 83 (10):1598-1610, 2001) note that the effects of wear debris on macrophage and monocyte cell lines have been studied extensively. Huo and Cook point to recent studies showing that osteoblasts are involved in the phagocytosis of wear debris and may contribute to the development and progression of osteolysis.

It is also possible that osteoclasts play a major role in osteolysis.

According to Huo and Cook, it has been found that metallic debris affects osteoblast function through two distinct mechanisms: (a) a direct negative effect on cellular function by the phagocytosis itself, and (b) an effect, mediated through cytokines, causing downregulation of procollagen alpha-1 gene expression along with decreased cell proliferation. It has been further demonstrated that osteoblasts stimulated by particulate debris produced interleukin-6 and prostaglandin E2, leading to the activation of osteoclast function.

Huo and Cook report another study demonstrating a direct, deleterious effect upon cellular function by the synovial fluid around loose total hip prostheses.

Huo and Cook also report that bone resorption by osteoclasts can be a result of an imbalance in the regulation of cellular activities, differentiation, proliferation, or survival. For example, a thirtyfold increase in cellular differentiation of cultured osteoclasts that were exposed to titanium particles was found, resulting in measurably increased bone resorption.

While considerable efforts have been made to contain wear debris, the present inventors have recognized a need for prosthetic implant devices having improved arrangements for containing wear debris.

### SUMMARY OF THE INVENTION

According to the teachings of the present invention there is provided a prosthetic implant device designed to provide protection against debris-related dysfunction when implanted in a living body of a human or an animal, the prosthetic implant device including: (a) a ball-and-socket prosthesis including at least: (i) a ball component having a first articulating surface, and (ii) a socket component having a second articulating surface, wherein, in a working configuration, the socket component is adapted to at least partially receive the ball component, to form an articulating, weight-bearing region disposed directly between the surfaces, and the first surface is adapted to move in a relative motion with respect to the second surface; (b) a sealing arrangement, associated with the prosthesis, the arrangement having a sealing member having a sealing face disposed substantially opposite, and abutting a portion of the first articulating surface, to form a sealing region adapted to obstruct a discharge of debris particles disposed directly between the articulating surfaces, from the ball-and-socket prosthesis, via the sealing region, and (c) at least one chamber, associated with the prosthesis, and adapted to receive and contain the debris particles.

According to another aspect of the present invention there is provided a prosthetic implant device designed to provide protection against debris-related dysfunction when implanted in a living body of a human or an animal, the prosthetic implant device including: (a) a prosthesis, such a ball-and-socket prosthesis, including: (i) a first component having a first articulating surface, and (ii) a second component having a second articulating surface, wherein, in a working configuration, the second component is adapted to at least partially juxtapose or superimpose with the first component, to form an articulating, load-bearing region disposed between or directly between the surfaces, and the first surface is adapted to move in a relative motion with respect to the second surface; and (b) a sealing arrangement, associated with the prosthesis, the arrangement including a sealing member having a sealing face disposed substantially opposite the first articulating surface, and abutting a portion thereof, to form a sealing region with the first articulating surface.

According to further features in the described preferred embodiments, the chamber associated with the prosthesis is disposed outside a perimeter of the articulating region.

According to further features in the described preferred embodiments, the chamber is disposed outside the ball and/or socket components, for example, external to the acetabular cup of the prosthesis.

According to still further features in the described preferred embodiments, the chamber is adapted to receive and contain debris particles having a short characteristic dimension of at least 15 micrometers, at least 10 micrometers, at least 5 micrometers, at least 2 micrometers, or at least 0.5 micrometers.

According to still further features in the described preferred embodiments, the prosthetic device further includes a fastening mechanism adapted to secure at least a portion of the sealing arrangement in a substantially fixed position with respect to the socket component.

According to still further features in the described preferred embodiments, the fastening mechanism is adapted to exert a superatmospheric pressure against the sealing face, towards the first articulating surface or substantially perpendicular thereto.

According to still further features in the described preferred embodiments, the fastening mechanism exerts a superatmospheric pressure against the sealing face, to urge the sealing face against the first articulating surface.

According to still further features in the described preferred embodiments, the fastening element is attached to the socket component.

According to still further features in the described preferred embodiments, the fastening element is integral or unitary with the socket component.

According to still further features in the described preferred embodiments, a first end of the fastening element is secured within a recess of the socket component.

According to still further features in the described preferred embodiments, the sealing member is at least partially disposed between the chamber and the first articulating surface.

According to still further features in the described preferred embodiments, the sealing arrangement includes a flow-guiding member having a first face, distinct from the sealing face, the first face disposed substantially opposite at least a portion of the first articulating surface, the flow-guiding member disposed at a finite distance from the first articulating surface to form a guiding passageway enabling fluid communication between the sealing face and the articulating region.

According to still further features in the described preferred embodiments, the sealing arrangement includes a flow-guiding member having a first face, distinct from the sealing face, the first face disposed substantially opposite at least a portion of the first articulating surface, the flow-guiding member disposed at a finite distance from the first articulating surface to form a guiding passageway between the sealing face and the articulating region, the guiding passageway having a cross-section enabling a debris-laden fluid containing the debris particles within the articulating region to traverse the guiding passageway to contact the sealing face.

According to still further features in the described preferred embodiments, the sealing arrangement includes a flow-guiding member having a first face, distinct from the sealing face, the first face disposed substantially opposite at least a portion of the first articulating surface, the flow-guiding member disposed at a finite distance from the first articulating surface to form a guiding passageway between the sealing face and the articulating region, the guiding passageway having a cross-section enabling a liquid external to the ball-and-socket prosthesis to traverse the guiding passageway and to enter the articulating region.

According to still further features in the described preferred embodiments, the flow-guiding member is fixed with respect to the first articulating surface, whereby a gap width between the flow-guiding member and the first articulating surface is at least 10 micrometers, at least 20 micrometers, at least 30 micrometers, at least 50 micrometers, or at least 70 micrometers.

According to still further features in the described preferred embodiments, the flow-guiding member is fixed with respect to the first articulating surface whereby a gap width between the flow-guiding member and the first articulating surface is less than 1000 micrometers, less than 500 micrometers, less than 300 micrometers, or less than 200 micrometers.

According to still further features in the described preferred embodiments, a first passageway to the at least one chamber is disposed between the first face and the sealing face, the first passageway adapted to at least intermittently enable fluid communication between the first articulating surface and the chamber.

According to still further features in the described preferred embodiments, the chamber is disposed and configured to receive and contain debris disposed between the first face and the sealing face.

According to still further features in the described preferred embodiments, a fastening element urges the sealing member against the flow-guiding member.

According to still further features in the described preferred embodiments, the sealing member has a substantially full cross-section, whereby debris and liquid are obstructed from passing therethrough.

According to still further features in the described preferred embodiments, between articulating surfaces is defined a first gap width (W₁), between the first articulating surface and the first face is defined a second gap width (W₂), and wherein a ratio of the second gap width to the first gap width is at least 2, at least 3, at least 5, or at least 10.

According to still further features in the described preferred embodiments, the first gap width is measured at an interface with the first face.

According to still further features in the described preferred embodiments, the sealing region substantially completely surrounds the first articulating surface.

According to still further features in the described preferred embodiments, the sealing member is spring-loaded against the first articulating surface.

According to still further features in the described preferred embodiments, the sealing arrangement is adapted whereby, when the first articulating surface moves in relative motion with respect to the second articulating surface, the debris is urged towards the chamber.

According to still further features in the described preferred embodiments, the ball-and-socket prosthesis is a hip prosthesis.

According to still further features in the described preferred embodiments, the sealing face is made of a biocompatible material, typically an elastomer, having a Shore A hardness in a range of 35 to 80, 40 to 75, 45 to 70, or 50 to 70.

According to still further features in the described preferred embodiments, the sealing arrangement includes a backbone or wall, secured on a first side by a fastening element or mechanism, and operatively connected, on a second side, to the sealing face.

According to still further features in the described preferred embodiments, the chamber is at least partially defined by an interior surface of the backbone or wall.

According to still further features in the described preferred embodiments, the backbone or wall is spring-loaded to deliver a pressure on the sealing face, to urge the sealing face towards the first articulating surface.

According to still further features in the described preferred embodiments, a total length of a flow pathway within the chamber is at least 10 mm, at least 25 mm, at least 50 mm, or at least 100 mm.

According to still further features in the described preferred embodiments, a total length of a flow pathway disposed between an inlet and an outlet of the chamber is at least 10 mm, at least 25 mm, at least 50 mm, or at least 100 mm. Typically, the outlet is distinct from the inlet.

According to still further features in the described preferred embodiments, a debris pathway including the first passageway and the chamber is at least partially defined by at least one of the articulating surfaces.

According to still further features in the described preferred embodiments, a debris pathway including the first passageway and the chamber is at least partially defined by the sealing face and at least one of the articulating surfaces.

According to still further features in the described preferred embodiments, the sealing arrangement is designed and configured whereby, when implanted in the body, the debris particles within the chamber are substantially fluidly isolated from white blood cells of the body.

According to still further features in the described preferred embodiments, a volume of the at least one chamber is at least 0.5 ml, at least 1.0 ml, at least 1.5 ml, at least 2.0 ml, or at least 2.5 ml.

According to still further features in the described preferred embodiments, the prosthetic device further includes a debris diversion assembly adapted to mechanically divert the debris particles from a vicinity of at least one of the articulating surfaces, towards the chamber.

According to still further features in the described preferred embodiments, the prosthetic device further includes a debris diversion assembly adapted to mechanically divert the debris particles away from a vicinity of the first articulating surface, towards the chamber.

According to still further features in the described preferred embodiments, the debris diversion assembly includes the first articulating surface and the sealing member.

According to still further features in the described preferred embodiments, a width of the first passageway is adapted to vary during relative motion between the articulating surfaces.

According to still further features in the described preferred embodiments, as the first articulating surface rotates downward with respect to the second articulating surface, the sealing face is urged downward therewith, whereby an opening to the first passageway is widened.

According to still further features in the described preferred embodiments, as the first articulating surface rotates upward with respect to the second articulating surface, the sealing face is urged upward therewith, whereby an opening to the first passageway is narrowed.

According to still further features in the described preferred embodiments, the first passageway is adapted to deliver a fluid to the at least one chamber, the fluid being laden with the debris particles.

According to still further features in the described preferred embodiments, the chamber has at least a second passageway adapted to withdraw the fluid from the at least one chamber, the fluid being debris-depleted with respect to the fluid laden with the debris particles.

According to still further features in the described preferred embodiments, within the chamber is disposed a porous medium adapted to trap the debris particles to produce a debris-depleted fluid.

According to still further features in the described preferred embodiments, the second articulating surface is adapted to at least partially receive the first articulating surface.

According to still further features in the described preferred embodiments, the second articulating surface has a concave contour adapted to receive or at least partially receive the first articulating surface.

According to still further features in the described preferred embodiments, the second surface, in a working configuration, is adapted to substantially contact the first surface.

According to still further features in the described preferred embodiments, in working configuration, the second surface is adapted to contact the first surface over substantially the entire articulating region.

According to still further features in the described preferred embodiments, at least one of the articulating surfaces is a substantially continuous articulating surface.

According to still further features in the described preferred embodiments, both of the articulating surfaces are substantially continuous articulating surfaces.

According to still further features in the described preferred embodiments, both of the articulating surfaces are substantially smooth, complementary articulating surfaces, typically over substantially the entire articulating region.

According to still further features in the described preferred embodiments, the articulating surfaces are substantially grooveless.

According to still further features in the described preferred embodiments, the articulating region is a sole articulating region between the first component and the second component.

According to still further features in the described preferred embodiments, the articulating region is a sole and substantially contiguously disposed articulating region between the first component and the second component.

According to still further features in the described preferred embodiments, the sealing arrangement and the second component are adapted, wherein, in a working arrangement, the sealing member abuts the second component.

According to still further features in the described preferred embodiments, a debris collection chamber is disposed and adapted, whereby, during articulation of the prosthesis, debris particles disposed directly between the articulating surfaces are received and contained therein.

According to still further features in the described preferred embodiments, the device may further include a debris diversion assembly adapted to mechanically divert the debris particles from a vicinity of the first articulating surface, or from a vicinity of at least one of the articulating surfaces, towards the chamber.

According to another aspect of the present invention there is provided a prosthetic implant device designed to provide protection against debris-related dysfunction when implanted in a living body of a human or an animal, the prosthetic implant device including: (a) a ball-and-socket prosthesis including at least: (i) a ball component having a first articulating surface, and (ii) a socket component having a second articulating surface, the device including any feature described, either individually or in combination with any feature, in any configuration.

According to another aspect of the present invention there is provided a method of operating a prosthetic implant device, substantially as described herein, the method including any feature described, either individually or in combination with any feature, in any configuration.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 provides a schematic, cross-sectional view of a prosthesis of the prior art;

FIG. 2 is a partial, schematic, cross-sectional view of a portion of a first exemplary embodiment of a prosthetic implant device according to the present invention;

FIG. 3A provides a schematic, perspective view of another exemplary embodiment of the inventive prosthetic implant device;

FIG. 3B is a schematic, cross-sectional, partially cut-open view of the prosthetic implant device of FIG. 3A;

FIG. 3C is a magnified view of a portion of the inventive sealing arrangement shown in FIG. 3B;

FIG. 4A provides a schematic, cross-sectional, partially cut-open view of another exemplary embodiment of a prosthetic implant device according to the present invention;

FIG. 4B provides a magnified view of a portion of FIG. 4A;

FIGS. 4C and 4D are magnified views of a portion of FIG. 4A, showing a disposition of the inventive sealing arrangement with respect to the articulating ball surface and with respect to the acetabular cup;

FIG. 5A provides a schematic, perspective view of another exemplary embodiment of a prosthetic implant device according to the present invention;

FIG. 5B is a schematic, cross-sectional, partially cut-open view of the prosthetic implant device provided in FIG. 5A;

FIG. 5C is a schematic, magnified view of a portion of the inventive sealing arrangement shown in FIG. 5B;

FIG. 5D is a schematic, expanded view of the interfacial arrangements within the inventive sealing arrangement shown in FIG. 5B;

FIG. 6A provides a partial, schematic, perspective, cut open view of a prosthetic implant device according to the present invention;

FIGS. 6B-6C provide magnified views of a portion of the prosthetic implant device shown in FIG. 6A;

FIG. 7A provides a schematic, perspective, cut open view of an exemplary debris-trapping arrangement according to the present invention;

FIG. 7B is another perspective, cut-open view of the debris-removing arrangement of FIG. 7A, in which the structure of a series of interconnecting passageways may be viewed;

FIG. 7C is a schematic ring structure that may be used to form the interconnecting passageways of FIG. 7B, and

FIGS. 8A-8C provide schematic, cross-sectional views of a sealing arrangement having a debris diversion assembly and a debris trapping arrangement, according to the present invention, wherein:

FIG. 8A shows a sealing member in a first, downwardly disposed position, such that a debris passageway is opened between adjacent surfaces of the sealing member and a flow-guiding member;

FIG. 8B shows the sealing member of FIG. 8A in a second, intermediate position, in which the passageway is closed at the entrance thereof by a surface of the sealing member abutting a surface of the flow-guiding member; and

FIG. 8C shows the sealing member of FIG. 8A in a third, upwardly disposed position, in which the passageway is closed along the length of the passageway by a surface of the sealing member abutting a surface of the flow-guiding member.

### DESCRIPTION OF THE PREFERRED EMBODIMENTS

The principles and operation of the prosthetic implant device according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

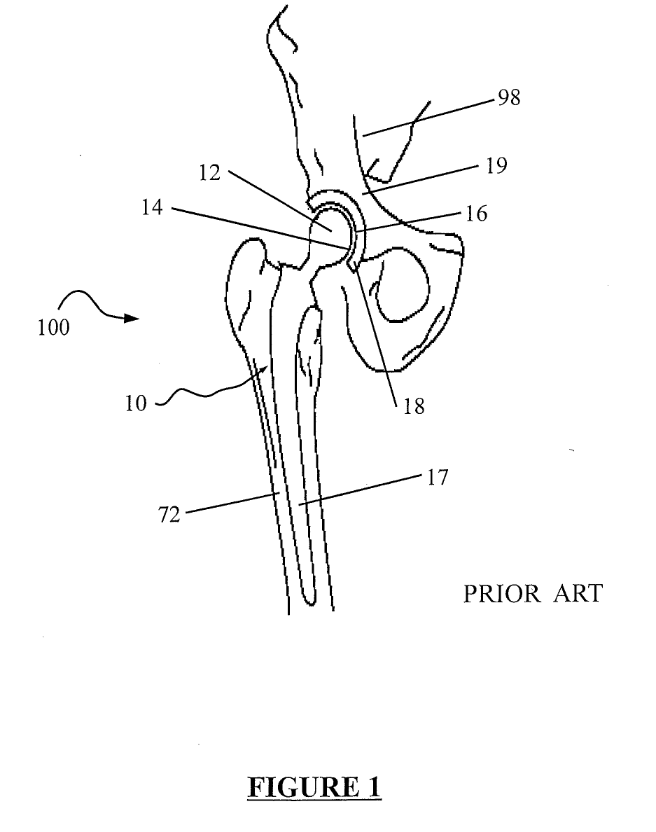

Referring generally to the drawings, FIG. 1 provides a schematic, cross-sectional view of a prosthetic implant device or prosthesis 100 of the prior art. An artificial femoral component or assembly 10 may include a first component of prosthesis 100, such as a femoral head 12, which is attached by way of a femoral neck to a prosthetic or femoral stem 17. Femoral head 12 has a first articulating surface 14, preferably contoured to largely complement a second articulating surface 16 of a second component of prosthesis 100, such as an acetabular cup 18. Articulating surface 14 and articulating surface 16 form at least a part of an artificial joint of prosthesis 100.

Femoral stem 17 is adapted to be inserted in a hollow, typically a substantially longitudinal hollow, produced in a femur 72 of the upper leg.

The second component or assembly of prosthesis 100, such as cup or acetabular cup 18, may be attached to tissue such as bone tissue. In the case of a hip joint, acetabular cup 18 is usually attached to a pelvic bone 19 known as the acetabulum, a part of the pelvis 98 where the human femoral head is located. Acetabular cup 18 may be attached to the bony acetabulum by various means, including gluing using a suitable cement such as a bone cement, using a press-fit technology preferably combined with induced bony ingrowth into cup 18, attaching cup 18 to the underlying bone tissue using screws or other securing hardware, various combinations of the above methods, or by other means known in the art.

As shown schematically in FIG. 1, first articulating surface 14 and second articulating surface 16 are juxtaposed to form the opposing, complementary, and typically substantially parallel, surfaces of prosthesis 100.

Acetabular cup 18 may include an outer cup, typically made of metal, and an inner cup, typically made of plastic. The inner cup may also be made of plastic, or of a second layer of metal. The outer cup may be designed and configured to attach to the underlying bone tissue, substantially as described hereinabove. An outer surface of the inner cup may be designed and configured to conform to an inner surface of the outer cup, while an inner surface of the inner cup makes up second articulating surface 16.

Without wishing to be limited by theory, one mechanism by which tissue decays in the vicinity of a prosthesis may be activated, or at least promoted, by the contact of prosthetic debris with cellular components within the synovial fluid.

By inhibiting fluid communication between cellular components within the synovial fluid present in, or produced in, a volume of body tissue disposed outside prosthesis 100, and the debris trapped within a volume of prosthesis 100, we believe that the lifetime of the prosthesis may be significantly prolonged. While various envelope-type arrangements may be implemented to inhibit this fluid communication, there may be various problems associated with such arrangements, including additional surgical procedures. Such envelope-type arrangements may be prone to aging, which may result in hardening or cracking of the envelope wall. A ruptured envelope might suddenly and disadvantageously expose a high concentration of debris to cellular components within the synovial fluid and to body tissue outside prosthesis 100.

Moreover, such envelope-type arrangements may restrict or impair joint motion, and may require, within the prosthesis, additional space, which is quite limited to begin with. Also, envelope-type arrangements may not prevent debris from re-entering the articulating surfaces, and possibly causing additional damage thereto. Envelope-type arrangements may also appreciably complicate surgical procedures.

We believe that it may be of particular advantage to overcome various disadvantages of various envelope-type arrangements by at least partially containing, or substantially completely containing, wear debris at or near the articulating surfaces, e.g., at the femoral head.

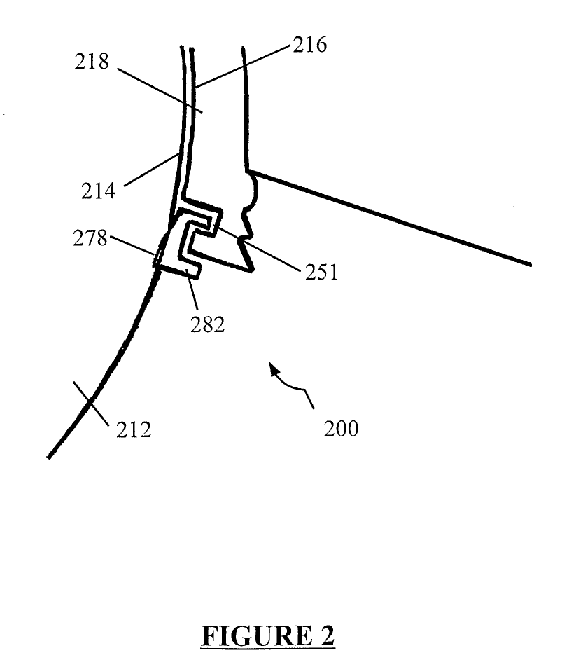

To this end, a partial, schematic, cross-sectional view of a portion of a first exemplary embodiment of an inventive prosthetic implant device 200 is provided in FIG. 2. While this and other exemplary embodiments provided herein pertain specifically to a femoral prosthesis, it is understood that these embodiments may broadly apply to other joints, typically ball-and-socket joints, in the anatomy of humans as well as those of various animals.

The artificial femoral component, including a first articulating surface 214, as well as a second articulating surface 216 of an acetabular cup 218, may be substantially identical to those provided hereinbelow (e.g., femoral component 310, first and second articulating surfaces 314, 316). A sealing member such as a spring-loaded sealing member or element 282 envelops and more typically, completely surrounds a femoral head 212. Sealing element 282 may be adapted to mount onto, or to be attached or juxtaposed to, acetabular cup 218.

In this exemplary embodiment, sealing element 282 is generally C-shaped or has a generally C-shaped section, in which a central section thereof forms a sealing face 278 that may be disposed generally along articulating surface 214 of femoral head 212. A first or top end of sealing element 282 may be received by a recess 251 in acetabular cup 218. A second or bottom end of sealing element 282 may fit around the bottom surface of acetabular cup 218. This end of sealing element 282 may contribute to the pressure exerted by sealing element 282 against articulating surface 214.

Sealing face 278 may have an angled contour so as to more closely impinge on articulating surface 214 at a bottom end of first face 278, i.e., at the distal end of first face 278, with respect to acetabular cup 218. This angled contour may at least partially inhibit debris from traversing sealing face 278 towards acetabular cup 218.

Sealing face 278 may be made of a biocompatible material having a Shore A hardness in a range of 35 to 80, such that sealing face 278 exhibits the requisite degree of conformability to articulating surface 214, and also exhibits the requisite degree of stiffness. More typically, the Shore A hardness of sealing face 278 is in a range of 40 to 75, or in a range of 45 to 70.

Sealing face 278 may consist of, or include, an elastomeric material characterized, inter alia, by high lubricity, reversible deformability and resilience, and physiological inertness. One such material may be a silicone polymer.

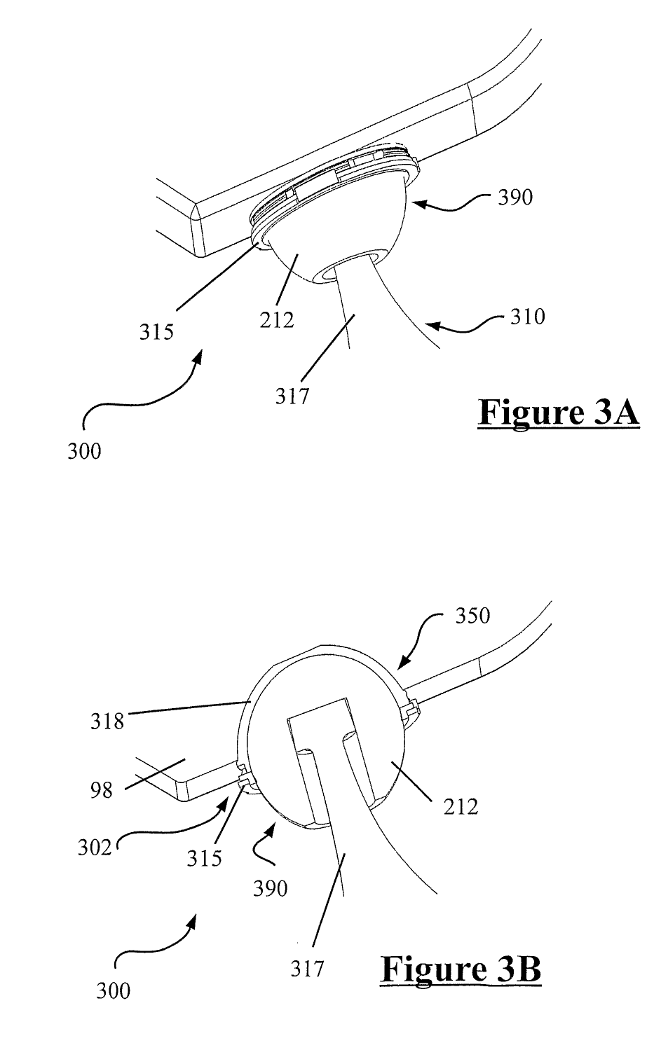

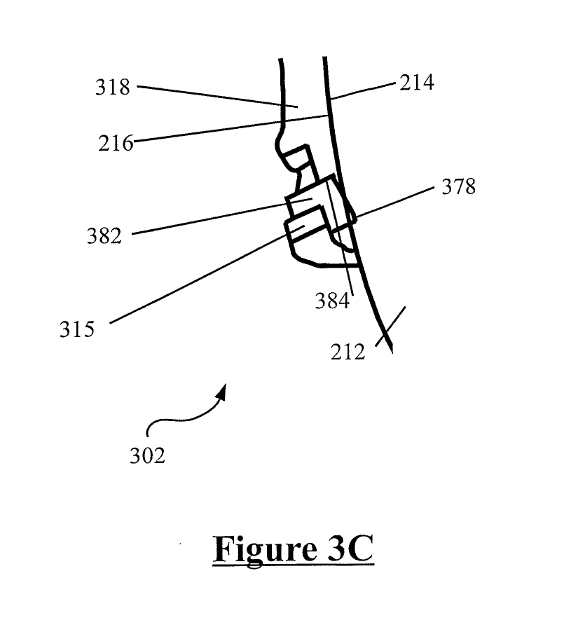

With reference now to FIGS. 3A-3C, FIG. 3A provides a schematic, perspective view of another exemplary embodiment of a prosthetic implant device 300 according to the present invention.

Prosthetic implant device 300 may include an artificial component such as an artificial femoral component 310, which may include a femoral stem 317, and which may include, or be associated with, a ball or head arrangement such as femoral head arrangement 390, which includes femoral head 212. Femoral head arrangement 390 may further include a fastening or pressuring member or arrangement, such as a fastening ring 315, which will be described in greater detail hereinbelow.

FIG. 3B provides a schematic, cross-sectional, partially cut-open view of prosthetic implant device 300. Prosthetic implant device 300 may include a second component of a prosthesis, such as an acetabular cup 318, which may be adapted to receive at least a portion of a femoral head 212. The structure for attaching acetabular cup 318 to a part of pelvis 98, and means for effecting the attachment, may be similar or substantially identical to those described hereinabove.

Within femoral head arrangement 390 are provided first and second articulating surfaces 214, 216 (shown, inter alia, with respect to FIG. 3C). These articulating surfaces may be juxtaposed or superimposed to form the opposing surfaces of an artificial joint 350.

Prosthetic implant device 300 may advantageously equipped with a seal or sealing arrangement 302, which may be adapted to mount onto femoral head 212. Alternatively or additionally, sealing arrangement 302 may be adapted to mount onto, or to be attached or juxtaposed to, acetabular cup 318.

FIG. 3C provides a magnified view of a portion of FIG. 3B, in which the structure of sealing arrangement 302 may be better viewed. Sealing arrangement 302 includes a sealing member 382, which may be generally L-shaped or have a generally L-shaped section, in which a first, sealing face 378 may be disposed generally along articulating surface 214 of femoral head 212, and a second face 384 may be disposed generally along an end surface of acetabular cup 318.

First face 378 may have a slightly angled contour so as to more closely impinge on articulating surface 214 at the bottom end of first face 378, i.e., at the distal end of first face 378, with respect to acetabular cup 318. This angled contour may at least partially inhibit debris from traversing sealing face 378 towards acetabular cup 318.

Fastening ring 315 may envelop, or completely surround, sealing face 378. Fastening ring 315 may be adapted to advantageously deliver a superatmospheric pressure to a circumference of sealing member 382, to urge sealing face 378 against or substantially against articulating surface 214. Alternatively or additionally, fastening ring 315 may be adapted to urge second face 384 against or substantially against the end surface of acetabular cup 318.

The physical and biological properties of sealing member 382 may be similar or substantially identical to those described hereinabove with regard to sealing face 278.

The advantages of this structure notwithstanding, the inventors have identified several potential problems associated therewith. The accumulation of debris between the articulating surfaces may accelerate wear in one or both of the articulating surfaces. This may negatively impact the longevity of the implant device. In some cases, catastrophic failure of the prosthesis may ensue. In addition, a sudden failure of the sealing arrangement may cause the accumulated debris to be released substantially at the same instant, which may have grave effects on the local body tissue and/or on various tissues within the body. Finally, debris particles arriving near the sealing arrangement may tend to collect in the area of the seal.

Thus, the present inventors have recognized a need for improved prosthetic implant devices that may successfully contend with some and preferably all of these identified problems.

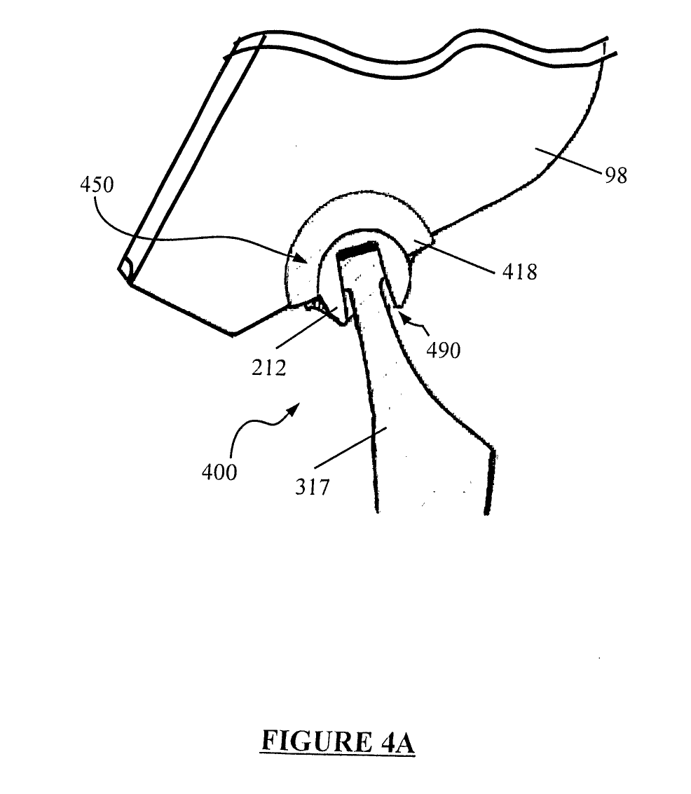

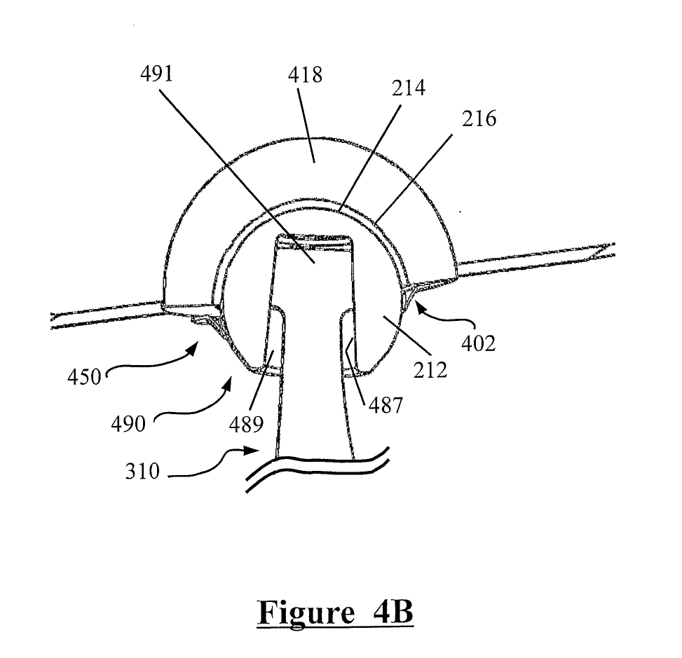

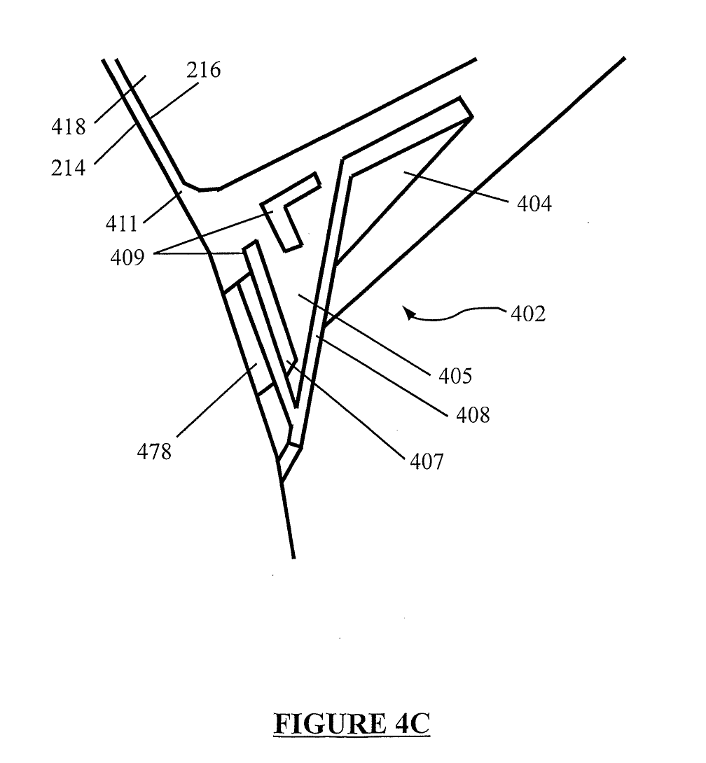

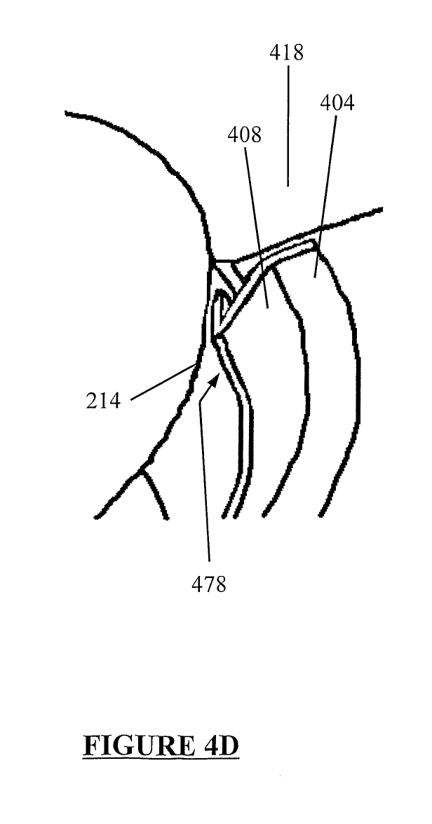

With reference now to FIGS. 4A-4D, FIG. 4A provides a schematic, cross-sectional, partially cut-open view of another exemplary embodiment of a prosthetic implant device 400 according to the present invention.

Prosthetic implant device 400 may include an artificial femoral component such as component 310, which may include femoral stem 317, and which may include, or be associated with, a femoral head arrangement 490 having femoral head 212.

An artificial joint 450 of implant device 400 may include a socket, such as an acetabular cup 418, which may be adapted to receive a portion of a femoral head 212. Acetabular cup 418 may be attached to tissue such as bone tissue, typically to the acetabulum. Acetabular cup 418 may advantageously be attached to the acetabulum by various means, some of which have been described hereinabove.

FIG. 4B provides a magnified view of a portion of FIG. 4A, in which femoral head arrangement 490 may be viewed with greater facility. Within femoral head arrangement 490, femoral head 212 is provided with a first articulating surface 214, preferably contoured to largely complement a second articulating surface 216 of a second component of a prosthesis, such as acetabular cup 418. Articulating surfaces 214 and 216 may be juxtaposed to form the opposing surfaces of artificial joint 450.

Femoral head 212 may have an inner wall 487 forming an opening or cavity 489 adapted to receive a portion or end 491 of femoral component 310. Preferably, inner wall 487 has a contour adapted to engage end 491 to achieve a snug fit. Such a fit may be achieved by providing at least one of end 491 and inner wall 487 with a substantially conic contour or otherwise narrowing contour, whereby when end 491 is introduced to cavity 489, the fit becomes more snug as end 491 extends longitudinally into cavity 489.

Artificial joint 450 may advantageously be equipped with a seal or sealing arrangement 402. Sealing arrangement 402 may be adapted to mount onto the acetabulum or on another portion of the pelvis. Alternatively, and as shown in the magnified views provided in FIG. 4C and in FIG. 4D, sealing arrangement 402 may be adapted to mount onto acetabular cup 418, by way of example, by means of a mounting area or strip 404. Various means may be used to attach sealing arrangement 402, including gluing, using securing hardware such as screws, tacks or nails, various combinations of these means, or by other means familiar to those of ordinary skill in the art.

The magnified view provided in FIG. 4C shows a disposition of sealing arrangement 402 with respect to articulating surface 214 and acetabular cup 418. Sealing arrangement 402 has a sealing face 478 disposed generally along articulating surface 214, and may be connected to mounting area 404 by means of a connecting backbone, wall, or strip 408.

Sealing face 478 is adapted to sealably contact at least a portion of articulating surface 214, to impede or obstruct passage of at least a portion of debris away from at least one of the articulating surfaces, or away from a divergence point 411 (i.e., the point at which the substantially parallel articulating surfaces begin to diverge) between articulating surfaces 214 and 216. By obstructing the debris, sealing arrangement 402 prevents the debris from leaving the area of articulating surfaces 214 and 216 and reaching other areas of the prosthetic implant device. Perhaps more significantly, sealing arrangement 402 prevents or largely inhibits the debris from contacting tissue adjacent to the prosthetic implant device, such as bone tissue of the acetabulum or various femoral tissues.

Sealing face 478 may be made of a biocompatible material having a Shore A hardness in a range of 35 to 80, such that sealing face 478 exhibits the requisite degree of conformability to articulating surface 214, and also exhibits the requisite degree of stiffness. More typically, the Shore A hardness of sealing face 478 is in a range of 40 to 75, in a range of 45 to 70, or in a range of 50 to 70.

Sealing face 478 may consist of, or include, an elastomeric material characterized, inter alia, by high lubricity, reversible deformability and resilience, and physiological inertness. One such material may be a silicone polymer.

The sealing efficacy of sealing face 478 may be enhanced by adapting backbone 408 to be spring-loaded, to deliver a pressure on sealing face 478, so as to urge sealing face 478 towards first articulating surface 214. To this end, sealing face 478 and backbone 408 may form a substantially V-shaped or U-shaped cross-section.

Sealing face 478 may exert a superatmospheric pressure that may be substantially normal to sealing face 478, and/or substantially normal to first articulating surface 214. Alternatively or additionally, the sealing arrangement may include at least one pressuring arrangement or element 407 adapted, and disposed with respect to sealing face 478, to urge face 478 towards articulating surface 214. Pressuring element 407 may include a spring adapted to deliver the pressure on sealing face 478.

Alternatively or additionally, when at least one of femoral head 212 and first articulating surface 214 is ferromagnetic, pressuring element 407 may advantageously include at least one magnetic element disposed, with respect to sealing face 478, to urge face 478 towards articulating surface 214.

Pressuring element 407 may be advantageously disposed between sealing face 478 and connecting backbone 408.

Sealing arrangement 402, and more particularly, sealing face 478, may be constructed in the fashion of a one-way valve, such that fluid on articulating surface 214 may flow along surface 214, past sealing face 478, in the direction of divergence point 411, while debris is obstructed or largely inhibited from traversing sealing face 478 in the opposite direction.

The debris obstructed by sealing arrangement 402 may be contained or trapped within at least one chamber or containing volume 405, which may be at least partially defined by at least one of articulating surface 214, sealing face 478, connecting backbone 408, and acetabular cup 418. Alternatively or additionally, containing volume 405 may be disposed within acetabular cup 418, in fluid communication with articulating surface 216.

Volume 405 may be equipped with at least one magnetic element or surface such as magnetic elements 409, which attract and capture ferromagnetic particles of the debris. Magnetic element 409 may be disposed in a substantially fixed position with respect to second articulating surface 216.

As shown, volume 405 is disposed outside a perimeter of the artificial joint, or outside a perimeter of the articulating region formed between first articulating surface 214 and second articulating surface 216.

The interior surface of sealing arrangement 402 (i.e., the surface in fluid communication with the volume adapted to receive and contain the debris), or portions thereof, may be provided with specific physical, mechanical, chemical, anti-microbial, and magnetic properties. The mechanical properties may include, by way of example, elasticity and mechanical strength. The physical and chemical properties may include, by way of example, physical affinity (e.g., absorption) and/or chemical affinity for metals or metal ions that may be present in the debris.

The sealing arrangement may extend solely between the ball and the socket, such that debris is contained therein, and is substantially inhibited from reaching the femoral neck or stem, and more importantly, from reaching areas of body tissue such as bone tissue of the acetabulum and femoral tissue. The sealing arrangement may be supplemented by various envelope arrangements.

Another preferred embodiment of the present invention pertains to the extent of the outer contour of the ball or head of the prosthesis. Conventional femoral heads, particularly those having a large diameter, may typically be generally hemispheric. In the ball-and-socket prosthetic device of the present invention, the exterior surface of the ball (e.g., femoral head 212) is preferably extended, such that sealing face 478 remains in contact with articulating surface 214, and debris in volume 405 remains securely contained, even during extreme articulations of the joint or prosthesis.

Thus, the outer contour of the ball or head may be appreciably more than hemispherical (i.e., having a 180° contour). Preferably, the outer contour extends at least 200°, at least 225°, at least 270°, or at least 300°. In terms of a ratio of the articulating surface area of the head to the articulating surface area of a hemisphere of identical diameter, the ratio may be at least 1.1, at least 1.25, at least 1.5, or at least 1.7.

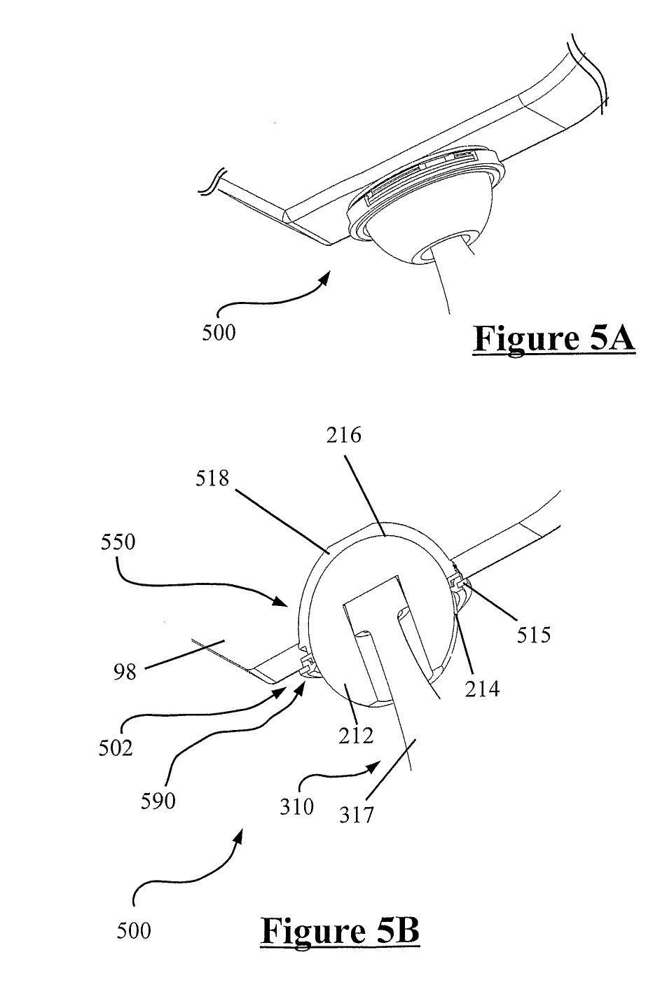

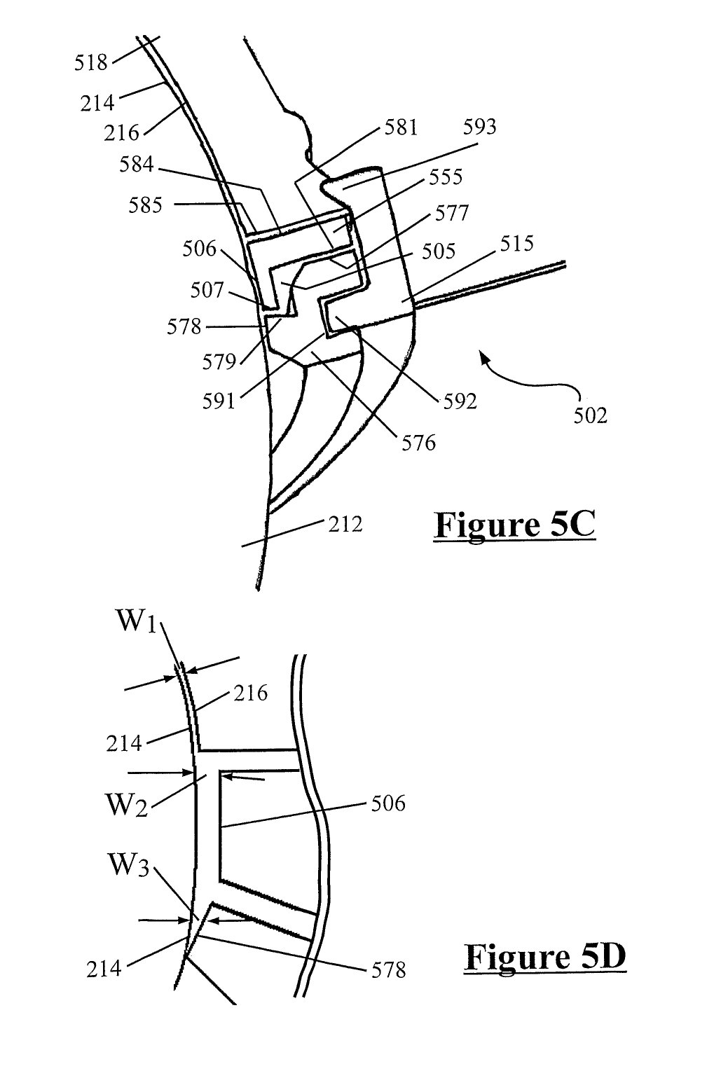

FIG. 5A provides a schematic, perspective view of another exemplary embodiment of a prosthetic implant device 500 according to the present invention. FIG. 5B provides a schematic, cross-sectional, partially cut-open view of prosthetic implant device 500. Broadly, prosthetic implant device 500 may largely resemble prosthetic implant device 300 described hereinabove. Prosthetic implant device 500 may have an artificial femoral component 310, which may include femoral stem 317, and includes, or is associated with, a femoral head arrangement 590. Femoral head arrangement 590 may include femoral head 212, and may include a fastening or pressuring member or arrangement, such as a fastening ring 515. Fastening ring 515 may serve a function similar or substantially identical to that of fastening ring 315, and is described in further detail hereinbelow.

The arrangement of the end of femoral component 310 within femoral head 212 may be similar or substantially identical to the arrangement described hereinabove. Prosthetic implant device 500 may include a second prosthetic component, such as an acetabular cup 518, which is adapted to receive a portion of femoral head 212. The structure for attaching acetabular cup 518 to a part of pelvis 98, and means for effecting the attachment, may be similar or substantially identical to those described hereinabove.

Within femoral head arrangement 590 are provided first and second articulating surfaces 214, 216, substantially as described hereinabove, inter alia with respect to FIG. 4B. These articulating surfaces may be juxtaposed or superimposed to form the opposing surfaces of an artificial joint 550.

Artificial joint 550 may be advantageously equipped with a seal or sealing arrangement 502. Sealing arrangement 502 may be adapted to mount onto femoral head 212. Alternatively, sealing arrangement 502 may be adapted to mount onto, or to be attached, urged against, or juxtaposed to, acetabular cup 518.

FIG. 5C provides a schematic, magnified view of a portion of FIG. 5B, in which the structure of sealing arrangement 502 may be better viewed. Sealing arrangement 502 may include a flow-guiding member 555. Member 555 may be generally L-shaped or have a generally L-shaped section. A first face 506 of member 555 may be disposed generally along articulating surface 214 of femoral head 212, and a second face 584, typically somewhat orthogonal to first face 506, may be disposed generally along an end surface 585 of acetabular cup 518.

First face 506 may have a slightly angled contour so as to more closely approach or impinge on (typically without contacting) articulating surface 214 at the bottom end of first face 506, i.e., at the distal end of first face 506, with respect to acetabular cup 518. This angled contour may contribute to a one-way valve action of sealing arrangement 502, in which fluid that contains debris disposed on or proximate to articulating surface 214, may flow along surface 214, past face 506, towards a sealing face 578 of a sealing member 576 (described in further detail hereinbelow), while debris may be obstructed or largely inhibited from traversing face 506 in the opposite direction.

In a presently preferred embodiment, the fluid itself may traverse face 506 in the opposite direction, i.e., towards acetabular cup 518.

Fastening ring 515 may at least partially envelop, or completely surround, face 506, to hold in place first face 506 proximate to, and generally opposite, articulating surface 214. Fastening ring 515 may be adapted to urge second face 584 against or substantially against end surface 585 of acetabular cup 518. Fastening ring 515 may be attached, anchored, or secured to acetabular cup 518, for example, by means of a recess in an outer surface of acetabular cup 518.

The physical and biological properties of first face 506, and more generally, flow-guiding member 555, may be similar to those described hereinabove with regard to sealing face 478. The rigidity or stiffness of face 506 may be sufficient to substantially maintain a contour of face 506 over the lifetime of the prosthetic device 500, even when moderate pressures are exerted on face 506. It may be of particular advantage for flow-guiding member 555 to exhibit a higher degree of mechanical absorbance (or a lower degree of stiffness) relative to sealing member 576. Thus, flow-guiding member 555 may have a Shore A hardness in a range of 30 to 75, and more typically, in a range of 30 to 70, in a range of 35 to 65, or in a range of 40 to 60.

Sealing arrangement 502 may include sealing member 576, which may be disposed generally underneath flow-confining member 555, distal to acetabular cup 518. Sealing member 576 may include a sealing face 578 disposed proximate to, and generally opposite, articulating surface 214. Sealing member 576 may also have a first top surface 579 disposed opposite a first bottom surface 507 of flow-confining member 555, proximate to articulating surface 214. A second top surface 577 of sealing member 576 may also be disposed opposite, and preferably abutting, a second bottom surface 581 of flow-confining member 555.

On a side of sealing member 576 that is generally distal to sealing face 578 may be disposed a groove or receptacle 591 adapted to receive a protruding end 592 of fastening ring 515. Fastening ring 515 may span sealing member 576 and flow-confining member 555, with a distal end 593 (with respect to protruding end 592) preferably being anchored in place. In FIG. 5C, distal end 593 is secured in place within a recess in a surface of acetabular cup 518.

Protruding end 592 of fastening ring 515 may exert a superatmospheric pressure towards distal end 593, whereby sealing member 576 may be urged towards, and flow-confining member 555 may be urged towards or against, end surface 585 of acetabular cup 518. Protruding end 592 of fastening ring 515 may exert a superatmospheric pressure towards articulating surface 214, whereby sealing member 576 may be urged towards, or against, articulating surface 214.

The pressure in these two directions may be achieved in various ways. For example, fastening ring 515 may be spring-loaded in the direction of articulating surface 214 and/or in the direction of end surface 585 of acetabular cup 518. Alternatively or additionally, sealing member 576 may be spring-loaded. For example, an inner circumference of sealing member 576 may be slightly less than an outer circumference of femoral head 212, whereby sealing face 578 may impinge or abut articulating surface 214 to substantially seal debris within sealing arrangement 502 or within artificial joint 550.

The debris obstructed by sealing arrangement 502 may be contained or trapped within at least one containing chamber or volume 505 that may be at least partially disposed between flow-confining member 555 and sealing member 576.

Volume 505 may be equipped with at least one magnetic member or surface adapted to attract and capture ferromagnetic particles of the debris. While various configurations may be possible (see, by way of example, magnetic element 409 in FIG. 4C), any interior surface within chamber 505 may be equipped with such a magnetic member.

Alternatively or additionally, the interior surfaces of volume 505 may be provided with specific physical, mechanical, chemical and/or biological properties. The mechanical properties may include, by way of example, elasticity and mechanical strength. The physical and chemical properties may include, by way of example, physical affinity (e.g., absorption) and/or chemical affinity for metals or metal ions that may be present in the debris.

Sealing arrangement 502 may extend solely between articulating surfaces 214 and 216, such that debris is contained therein, and may be at least partially inhibited or even substantially inhibited from reaching the femoral neck or stem, and more importantly, from reaching areas of body tissue such as bone tissue of the acetabulum and femoral tissue. Alternatively, and as shown in FIG. 5C, sealing arrangement 502 may be connected to end surface 585 of acetabular cup 518. In this configuration as well, debris may be contained within sealing arrangement 502, and may be at least partially inhibited or even substantially inhibited from reaching the femoral neck or stem, and from reaching areas of body tissue.

Sealing arrangement 502 may be supplemented by any of various envelope arrangements known in the art.

In another preferred embodiment, which may be best viewed in FIG. 5D, sealing arrangement 502 may be adapted whereby a distance or gap width W₂ between first face 506 and articulating surface 214 is at least equal to a gap width W₁ between articulating surfaces 214 and 216. At an interface between articulating surface 214, articulating surface 216, and first face 506, a ratio of gap width W₂ to gap width W₁ (adjacent to the interface) may be at least 2, at least 3, or at least 5 or more. Consequently, debris disposed between articulating surfaces 214 and 216 may not be obstructed or impeded by flow-guiding member 555, such that debris may not disadvantageously accumulate between surfaces 214 and 216. As a result, accelerated wear of particles generated between surfaces 214 and 216 of artificial joint 550 may be avoided.

Gap width W₂ between first face 506 and articulating surface 214 may be at least 10 micrometers, at least 20 micrometers, at least 30 micrometers, at least 50 micrometers, or at least 70 micrometers. However, an inordinately large gap may compromise the debris-guiding function of member 555. Thus, gap width W₂ may be less than 1000 micrometers, less than 500 micrometers, less than 300 micrometers, or less than 200 micrometers.

In another preferred embodiment, sealing arrangement 502 may be adapted whereby a distance or gap width W₃ between sealing face 578 and articulating surface 214 is less than gap width W₂. Gap width W₃ represents the largest distance between sealing face 578 and articulating surface 214. Consequently, particles generated between articulating surfaces 214 and 216, after passing by first face 506, may be advantageously directed into volume 505. Typically, sealing face 578 may sealingly contact articulating surface 214.

In a preferred embodiment, a fluid may be injected or introduced within any of the above-described sealing arrangements, e.g., via any of the above-described sealing members or elements, to provide lubricant to the sealing or articulating surfaces, to wash these surfaces, or for other purposes. The injection of fluid may be performed by passing a syringe, needle or the like through one or more sealing members. Similarly, the syringe may be used to withdraw fluid and particulate matter from within the sealing arrangement and/or from within a particle-containing volume such as volume 505.

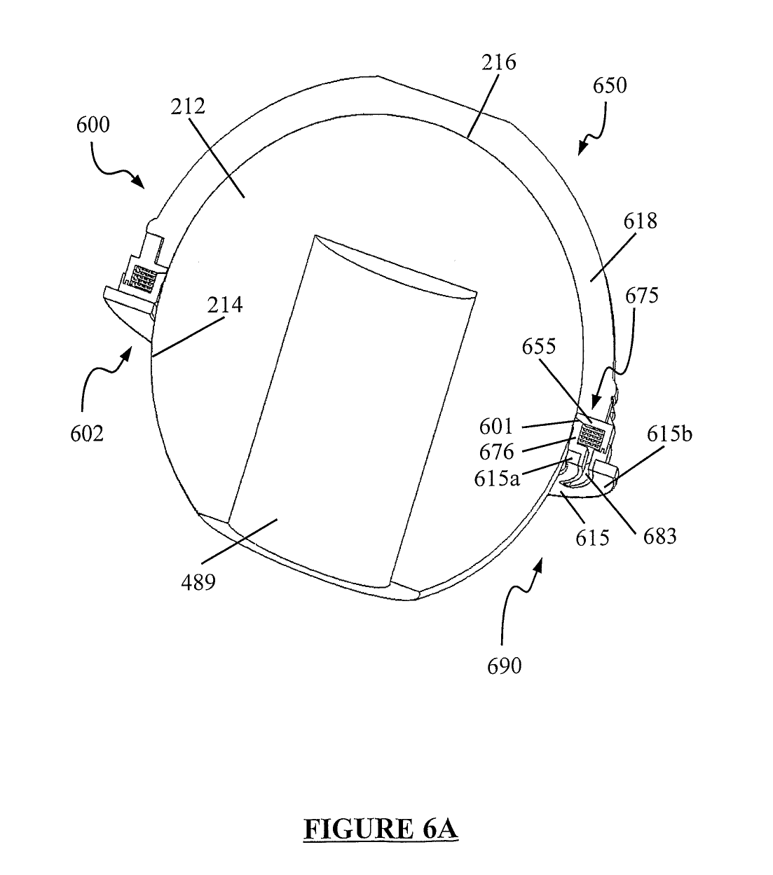

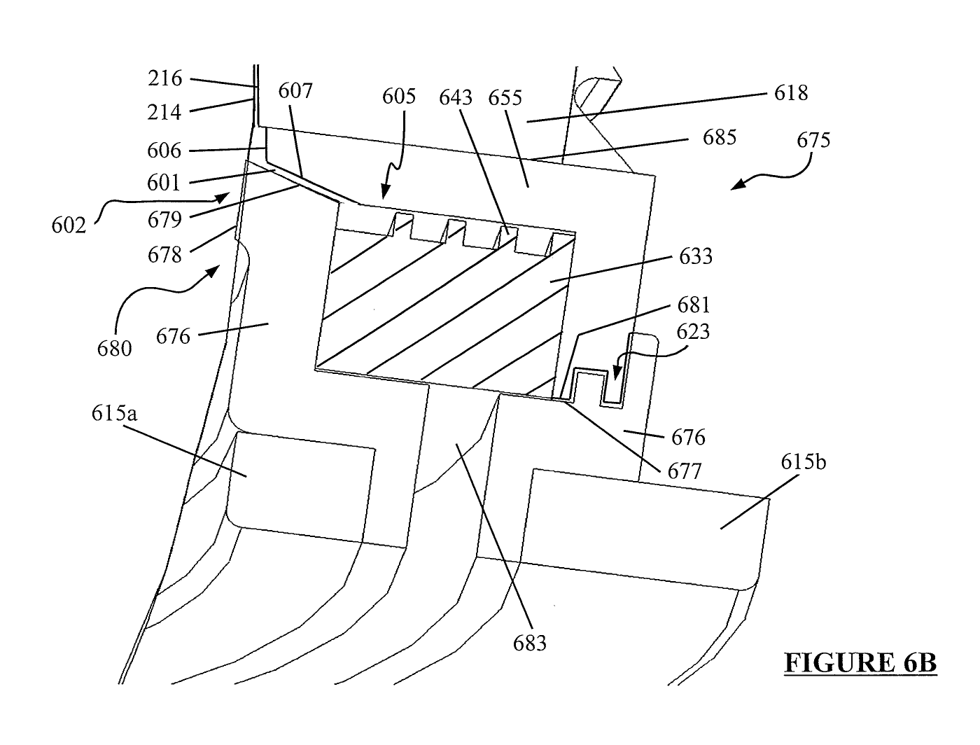

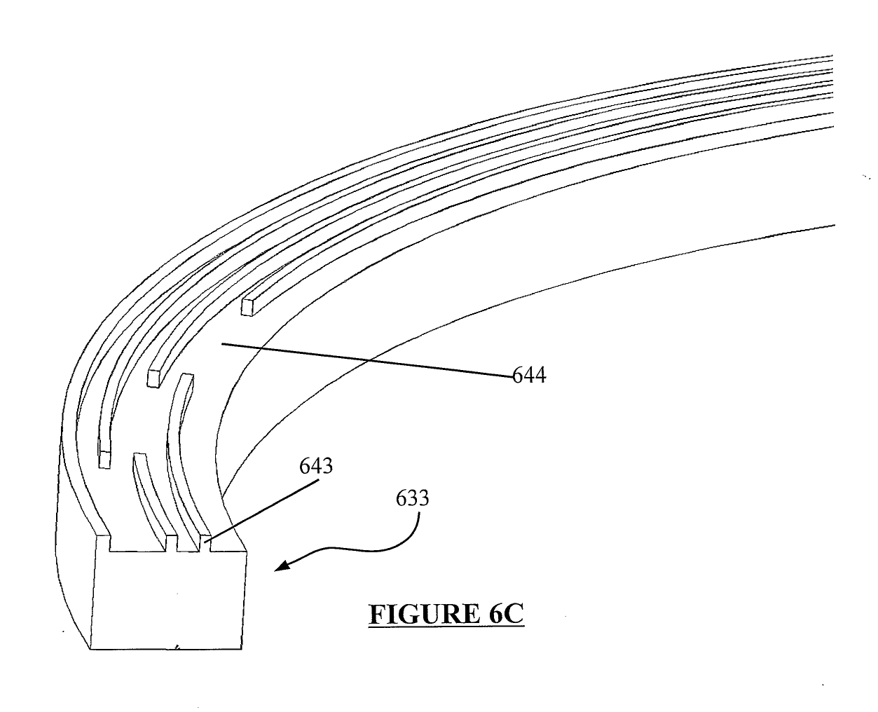

With reference now to FIGS. 6A-6C, FIG. 6A provides a partial, schematic, perspective, cut open view of a prosthetic implant device 600. FIGS. 6B-6C provide magnified views of the portion of prosthetic implant device 600 shown in FIG. 6A. Broadly, prosthetic implant device 600 may largely resemble the inventive prosthetic implant devices described hereinabove. Prosthetic implant device 600 has an artificial joint 650, in which first articulating surface 214 of femoral head 212 and second articulating surface 216 of acetabular cup 618 may be juxtaposed or superimposed to form the opposing joint surfaces, as shown in FIG. 6B. Femoral head 212 may have an opening or cavity 489 adapted to receive an end of a femoral component such as femoral component 310 (provided in FIG. 5B).

Associated with artificial joint 650 and a femoral head arrangement 690 is a debris-removing or debris-trapping arrangement such as debris-trapping arrangement 675, which may include a port, passageway or channel 601, and at least one chamber or containing volume 605 adapted to receive a relatively debris-rich fluid via passageway 601. Debris-trapping arrangement 675 may further include a port, passageway or channel 683 adapted to discharge a debris-depleted fluid that has passed through containing volume 605. As may be best viewed in FIG. 6A, passageway 683 may at least be partially surrounded or bounded by a sealing member 676 and by a fastening or pressuring member or arrangement, such as a fastening ring 615. In this exemplary embodiment, fastening ring 615 is a bifurcated ring having, with respect to femoral head 212, a proximate branch 615a and a distal branch 615b, and passageway 683 is longitudinally surrounded on opposite sides by branch 615a and branch 615b. Passageway 683 may be surrounded on one or more short sides by a non-bifurcated or continuous section of fastening ring 615, substantially as shown.

Artificial joint 650 is advantageously equipped with a seal or sealing arrangement 602. Sealing arrangement 602 may be adapted to mount onto femoral head 212. Alternatively, as may be seen in FIG. 6B, sealing arrangement 602 may be adapted to mount onto, or to be attached or juxtaposed to, acetabular cup 618.

FIG. 6B provides a cross-sectional view of debris-trapping arrangement 675 and of sealing arrangement 602. Sealing arrangement 602 includes a flow-guiding or flow-confining member 655 having a first face 606 disposed generally opposite articulating surface 214 of femoral head 212, and a second face that may be disposed generally along an end or bottom surface 685 of acetabular cup 618.

First face 606 may have a slightly angled contour so as to more closely approach or impinge on (typically without contacting) articulating surface 214 at the bottom end of first face 606, i.e., at the distal end of first face 606, with respect to acetabular cup 618. This angled contour may contribute to a one-way valve action of sealing arrangement 602, in which fluid that contains debris disposed on or proximate to articulating surface 214, may flow along surface 214, past face 606, towards a sealing face 678 of sealing member 676, while debris may be obstructed or largely inhibited from traversing face 606 in the opposite direction.

Sealing member 676 may be disposed generally underneath flow-confining member 655. Sealing face 678 is disposed proximate to, and generally opposite, articulating surface 214. Sealing member 676 may also have a first top surface 679 disposed opposite a first bottom surface 607 of flow-confining member 655. Sealing member 676 may also have a second top surface 677 disposed opposite, and preferably abutting, a second bottom surface 681 of flow-confining member 655. Second top surface 677 and second bottom surface 681 may be held in a relative fixed position, by means of a protrusion and recess arrangement 623, by way of example. Sealing member 676 may be adapted to exert a superatmospheric pressure upwards, against at least one bottom surface 607, 681, whereby sealing member 676 may be urged upwards, towards flow-confining member 655, such that flow-confining member 655 is urged towards end surface 685 of acetabular cup 618.

Channel 601 may be at least partially surrounded or bounded, longitudinally, by top surface 679 of sealing member 676 on a first side and by a first bottom surface 607 of flow-confining member 655 on an opposite side. The width of channel 601 may be adapted to vary during relative motion between articulating surfaces 214 and 216. As articulating surface 214 rotates downward with respect to articulating surface 216, sealing face 678 of sealing member 676 is urged downward therewith, typically by articulating surface 214. Since flow-confining member 655 remains substantially in place, or is urged downward less than top surface 679 of sealing member 676, channel 601 may be adapted to widen, thereby enabling debris-rich fluid (e.g., flowing from between articulating surfaces 214 and 216) to enter channel 601. Via channel 601, the debris-laden fluid may be introduced into chamber 605. It must be emphasized that as the debris-laden fluid passes by articulating surfaces 214 and 216 and flows downward between articulating surface 214 and first face 606, the debris-laden fluid may not continue along articulating surface 214, since sealing face 678 seals or obstructs the path along articulating surface 214. Thus, articulating surface 214 and sealing arrangement 602 (including sealing member 676 in particular) form at least a part of a debris-diversion assembly 680 adapted to divert or drive debris-laden fluid from its path along articulating surface 214, and into chamber 605. In this exemplary embodiment, debris-diversion assembly 680 is a mechanically driven debris-diversion assembly that is at least partially driven by the articulating motion of articulating surface 214.

As articulating surface 214 rotates upward, in an opposite direction, with respect to articulating surface 216, sealing face 678 may be urged upward, typically by articulating surface 214. Since flow-confining member 655 remains substantially in place, or is urged upward less than top surface 679 of sealing member 676, channel 601 may be adapted to narrow, or even substantially close, thereby inhibiting or at least partially inhibiting debris in chamber 605 from returning, via channel 601, towards the area between the articulating surfaces.

The narrowing of channel 601 may further serve to urge, into chamber 605, the debris-laden fluid disposed in channel 601.

Once disposed within chamber 605, debris from the debris-laden fluid may settle and possibly adhere to the walls of chamber 605, or to the walls of the passageways within chamber 605. Alternatively or additionally, chamber 605 may be equipped with a medium such as porous medium 633, which may be adapted to trap at least a portion of the debris particles within, and/or on a surface thereof, while enabling fluid to pass through. Thus, passageway 683 receives, and subsequently discharges, a debris-depleted fluid with respect to the debris-laden fluid flowing into chamber 605 via port 601.

Preferably, porous medium 633 may be structured whereby a cross-sectional area of a pore may narrow along the flow path through the pore. The pore inlet may have a cross-sectional area that is sufficient to receive the largest particles produced between the articulating surfaces.

Porous medium 633 may have a circular, ring-like structure, as shown in FIG. 6C. A top surface of porous medium 633 may have at least one ridge 643 adapted to partition or largely partition a receiving volume within chamber 605 into at least two sub-chambers. Ridge 643 may have intermittent open spaces 644 to enable distribution of the debris-laden fluid within at least two of such sub-chambers.

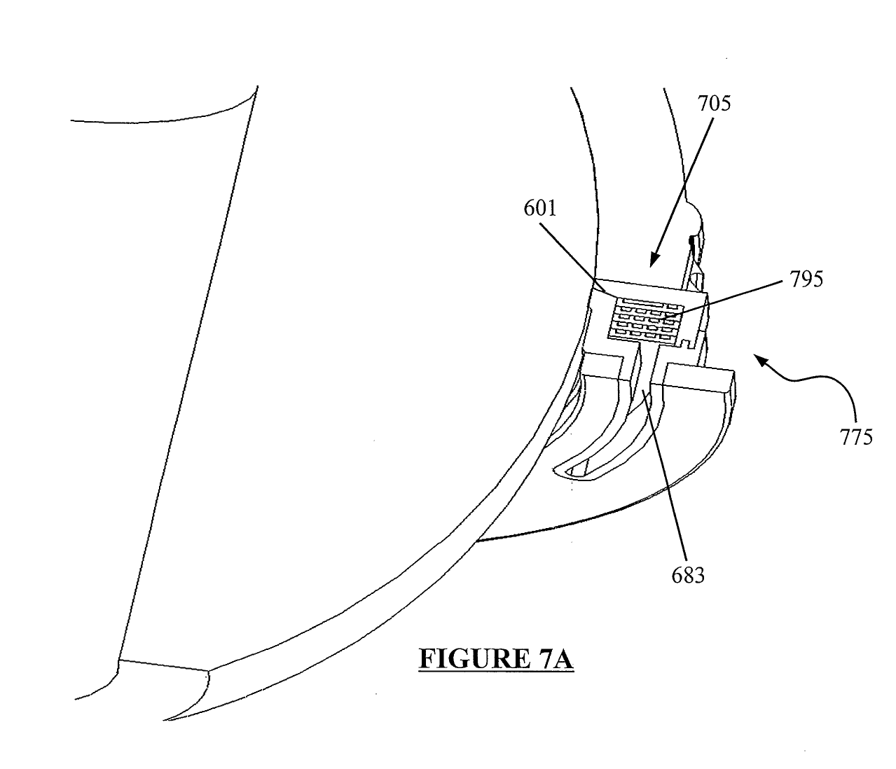

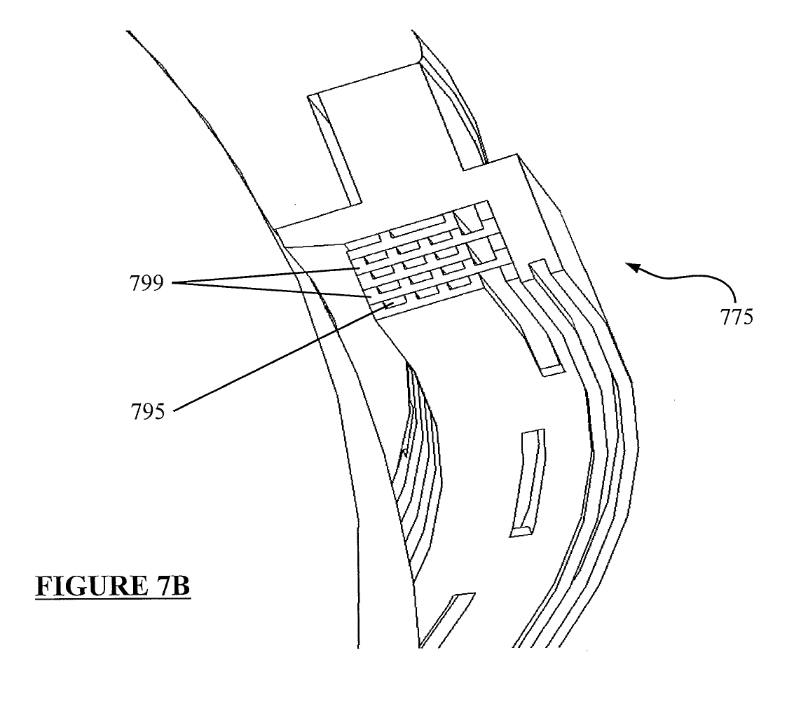

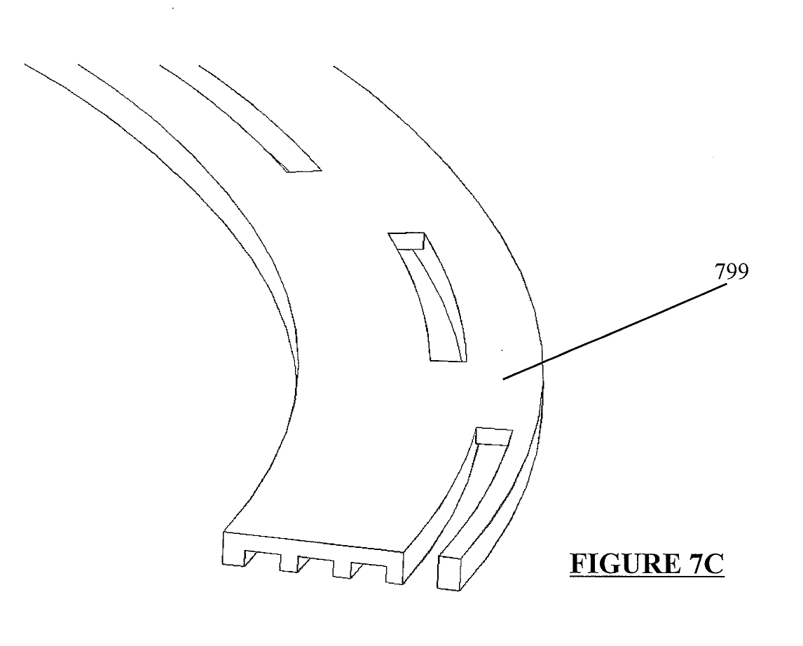

FIG. 7A provides a schematic, perspective, cut open view of another exemplary debris-removing or debris-trapping arrangement such as debris-removing arrangement 775, according to the present invention. Debris-removing arrangement 775 may be disposed within a prosthetic implant device such as a prosthetic implant device 600 described hereinabove. FIG. 7B provides another perspective, cut-open view of debris-removing arrangement 775, in which the structure of a series of interconnecting channels or passageways 795 may be viewed. This structure may be constructed from a plurality of rings such as ring 799, shown in perspective view in FIG. 7C.

With collective reference now to FIGS. 7A-7C, debris-laden fluid may be introduced into chamber 705 via channel 601. Within a chamber 705, which may be substantially identical to chamber 605 provided in FIG. 6B, the debris-laden fluid may be subjected to a long flow path, e.g., a flow path through a series of interconnecting channels or passageways 795. Debris from the debris-laden fluid may settle and possibly adhere to the walls of chamber 705, or, more typically, to the walls of passageways 795 within chamber 605. For a debris flow pathway from a first passageway of passageways 795 until the beginning or top of passageway 683, a total length of a flow pathway within the chamber is at least 10 mm, at least 25 mm, at least 50 mm, or at least 100 mm.

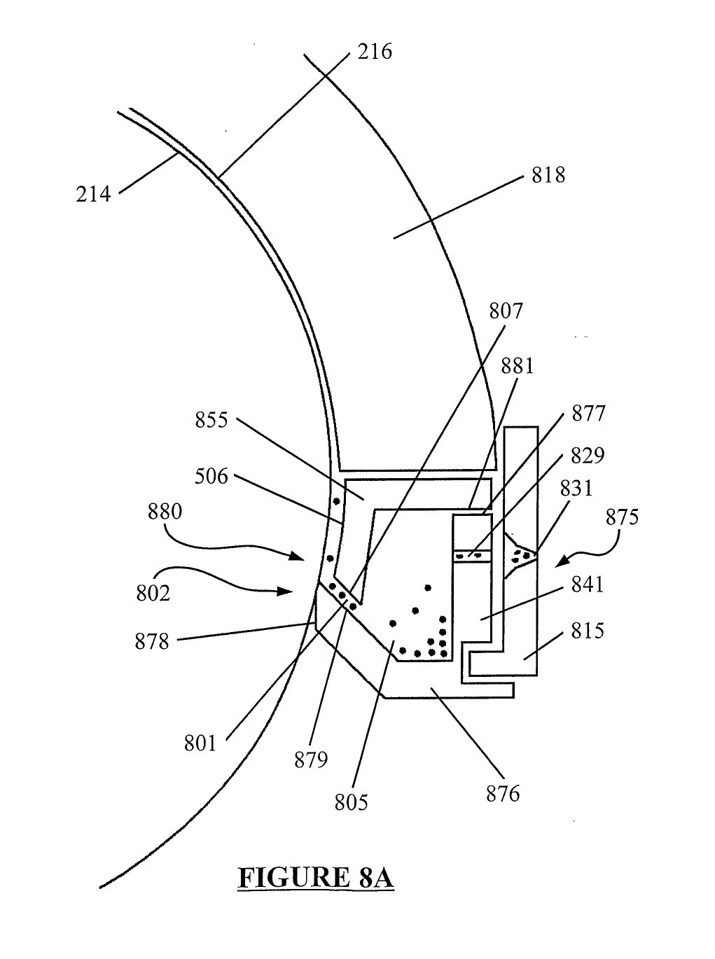

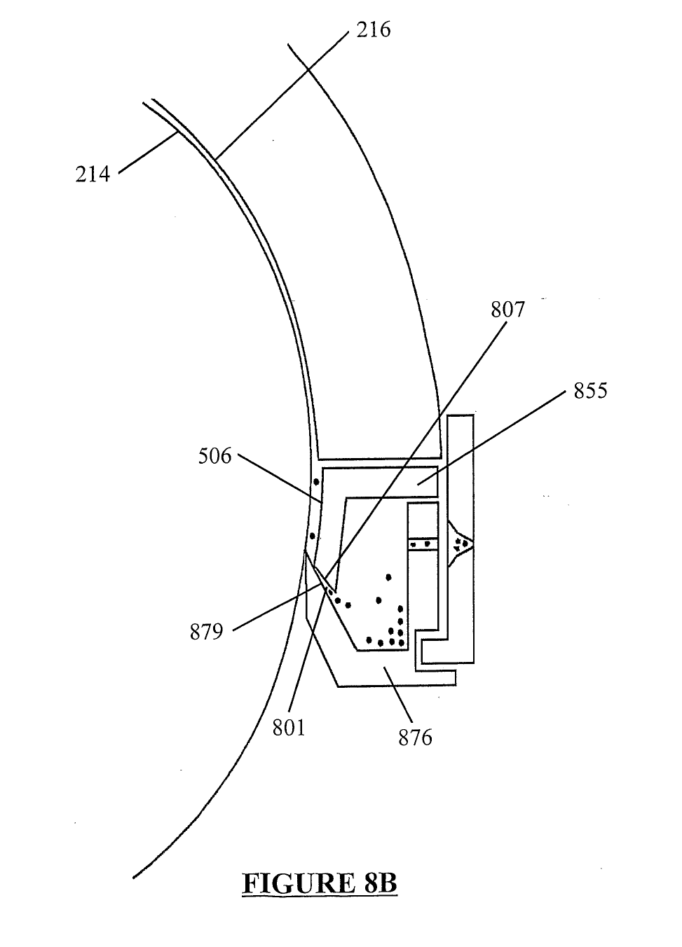

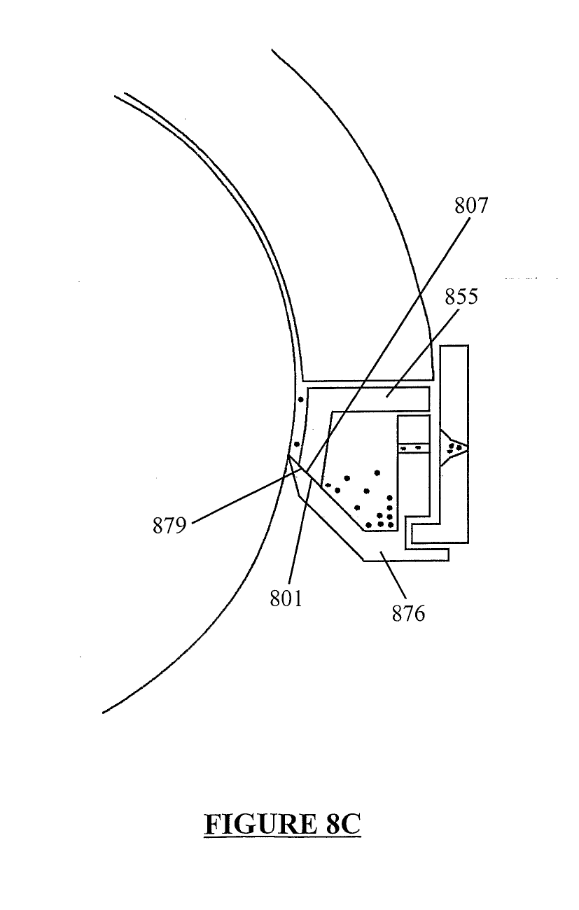

With reference now to FIGS. 8A-8C, FIGS. 8A-8C provide schematic, cross-sectional views of a sealing arrangement 802 having a mechanical debris diversion arrangement or assembly 880 and a debris trapping arrangement 875, according to an exemplary embodiment of the present invention. Broadly, various aspects of these arrangements may largely resemble the arrangements described hereinabove.

FIG. 8A shows a sealing member 876 in a first, downwardly disposed position, wherein a debris port, passageway or channel 801 is in an open position between adjacent surfaces of sealing member 876 (top surface 879) and a flow-guiding member 855 (bottom surface 807).

A fastening ring 815 may envelop, or completely surround, a sealing face 878 of sealing member 876. Fastening ring 815 may be adapted to advantageously deliver a superatmospheric pressure to a circumference of sealing member 876, to urge sealing face 878 against or substantially against articulating surface 214. Alternatively or additionally, fastening ring 815 may be adapted to urge an arm 841 of sealing member 876 whereby a second face 877 on arm 841 is urged against a second bottom surface 881 of flow-guiding member 855. In turn, bottom surface 881 may press against or substantially against an end or bottom surface of acetabular cup 818 to achieve sealing in the vertical direction.

The physical and biological properties of sealing member 876 may be similar or substantially identical to those described hereinabove.

Debris, typically along with fluid, may be mechanically driven through passageway 801 (as shown in FIGS. 8B-8C and described hereinbelow), and into a chamber such as chamber 805. Chamber 805 may be largely, or at least partially defined, by surfaces of sealing member 876 and member 855.

Debris and fluid may accumulate within chamber 805. Chamber 805 may form at least a part of debris trapping arrangement 875. It may be advantageous, however, to have an alternative or additional arrangement that enables fluid such as a lubricant liquid (e.g., synovial fluid) to be removed, or selectively removed.

As shown in exemplary fashion in FIG. 8A, within arm 841 of sealing member 876 is disposed at least one aperture or passageway 829 through which may traverse debris particles and liquid. Passageway 829 may be lined up with, or otherwise fluidly communicate with, a passageway 831 disposed in fastening ring 815. Passageway 831 may have a narrow outlet, whereby debris particles traversing passageway 831 may readily be trapped by the outlet, and/or by particles previously contained or trapped by the outlet. Also, debris particles traversing passageway 831 may adhere to the inner wall of passageway 831, and/or to other particles disposed therein.

One skilled in the art will appreciate that fastening ring 815 may consist of various biocompatible materials, including various metals or alloys, and that passageway 829 within arm 841 of sealing member 876 may simply be a hole, or may have a structural wall. It will be further appreciated that in the interest of clarity, passageway 829 is shown as if arm 841 is in a compressed or loaded state.

FIG. 8B shows sealing member 876 in a second, intermediate position, in which passageway 801 is closed at the entrance thereof by surface 879, which is urged against surface 807 of flow-guiding member 855. At this stage, the contents of passageway 801 may be sealed, or at least partially sealed, from the passageway (between surface 214 and surface 506) leading back to the articulating region of the prosthesis.

FIG. 8C shows sealing member 876 in a third, upwardly-disposed position, in which passageway 801 may be closed along a portion (typically most or all) of the length of passageway 801, by surface 879, which is urged against surface 807 of flow-guiding member 855. At this stage, the contents of passageway 801 are typically sealed from the passageway leading back to the articulating region of the prosthesis.

Thus, the instant invention may provide a mechanism adapted to guide debris away from the articulating region of the prosthesis, away from the articulating surface of the ball (e.g., a femoral head) and to direct the debris into a debris trap or chamber. This may be preferably accomplished without accumulation, or without substantial accumulation, of debris between the articulating surfaces.

The instant invention may further provide a mechanism whereby trapped or contained debris particles may be obstructed or substantially completely and unilaterally or irreversibly inhibited from returning to a position between the articulating surfaces.

The instant invention may further provide a debris trapping arrangement having a mechanism for removing fluid from the chamber, the fluid being debris-depleted with respect to the debris-laden fluid introduced to the chamber.

As used herein in the specification and in the claims section that follows, the term “contiguous”, “contiguously disposed”, or the like, with respect to an articulating region within a prosthetic device, refers to an articulation area that is connected in space, without a break, such as a groove or channel.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 10 should be considered to have specifically disclosed subranges such as from 1 to 2, from 1 to 5, from 1 to 8, from 3 to 4, from 3 to 8, from 3 to 10, etc., as well as individual numbers within that range, for example, 1, 2, 3, 4, 5, 6, 7, 8, 9, and 10. This applies regardless of the breadth of the range.

Similarly, the terms “at least”, “exceeds”, and the like, followed by a number (including a percent or fraction), should be considered to have specifically disclosed all the possible subranges above that number, as well as individual numerical values above that number. For example, the term “at least 75” should be considered to have specifically disclosed subranges such as 80 and above, 90 and above, etc, as well as individual numbers such as 85 and 95.

Similarly, the terms “less than”, “below”, and the like, followed by a number (including a percent or fraction), should be considered to have specifically disclosed all the possible subranges below that number, as well as individual numerical values below that number. For example, the term “below 75” should be considered to have specifically disclosed subranges such as 70 and below, 60 and below, etc, as well as individual numbers such as 65 and 50.

Whenever a numerical range is indicated herein, the range is meant to include any cited numeral (fractional or integral) within the indicated range. The phrase “ranging/ranges between” a first number and a second number and “within a range of” a first number to a second number, and the like, are used herein interchangeably and are meant to include the first and second indicated numbers and all the fractional and integral numerals therebetween.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-56. (canceled)

57. A prosthetic implant device designed to provide protection against debris-related dysfunction when implanted in a living body of a human or an animal, the prosthetic implant device comprising:
(a) a ball-and-socket prosthesis including at least:
(i) a ball component having a first articulating surface, and
(ii) a socket component having a second articulating surface,
wherein, in a working configuration, said socket component is adapted to at least partially receive said ball component, to form an articulating region disposed directly between said surfaces, and said first surface is adapted to move in a relative motion with respect to said second surface;
(b) a sealing arrangement, associated with said prosthesis, said arrangement including a sealing member having a sealing face disposed substantially opposite said first articulating surface, and abutting a portion thereof, to form a sealing region adapted to obstruct a discharge of debris particles disposed directly between said articulating surfaces, from said ball-and-socket prosthesis, via said sealing region, and
(c) at least one chamber, associated with said sealing arrangement, and adapted to receive and contain said debris particles.

58. The prosthetic device of claim 57, said chamber disposed outside said articulating region.

59. The prosthetic device of claim 57, further comprising a fastening mechanism adapted to secure at least a portion of said sealing arrangement in a substantially fixed position with respect to said socket component, said fastening mechanism is adapted to exert a superatmospheric pressure against said sealing face, towards or substantially perpendicular to said first articulating surface.

60. The prosthetic device of claim 59, wherein said sealing region substantially completely surrounds said first articulating surface, said fastening element is attached to said socket component, said fastening element is integral or unitary with said socket component, and first end of said fastening element is secured within a recess of said socket component, and said sealing face is made of a biocompatible material having a Shore A hardness in a range of 35 to 80.

61. The prosthetic device of claim 57, wherein said sealing member is at least partially disposed between said chamber and said first articulating surface.

62. The prosthetic device of claim 57, wherein said sealing arrangement includes a flow-guiding member having a first face, distinct from said sealing face, said first face disposed substantially opposite at least a portion of said first articulating surface, said flow-guiding member disposed at a finite distance from said first articulating surface to form a guiding passageway enabling fluid communication between said sealing face and said articulating region.

63. The prosthetic device of claim 62, wherein a first passageway to said at least one chamber is disposed between said first face and said sealing face, said first passageway adapted to at least intermittently enable fluid communication between said first articulating surface and said chamber.

64. The prosthetic device of claim 62, wherein between articulating surfaces is defined a first gap width (W₁), between said first articulating surface and said first face is defined a second gap width (W₂), and wherein a ratio of said second gap width to said first gap width is at least 2, at least 3, or at least 5.

65. The prosthetic device of claim 57, wherein said sealing member is spring-loaded against said first articulating surface.

66. The prosthetic device of claim 57, said sealing arrangement adapted whereby, when said first articulating surface moves in said relative motion with respect to said second articulating surface, said debris is urged towards said chamber.

67. The prosthetic device of claim 57, wherein a total length of a flow pathway disposed between an inlet and an outlet of said chamber is at least 10 mm, at least 25 mm, at least 50 mm, or at least 100 mm, wherein said outlet is distinct from said inlet.

68. The prosthetic device of claim 57, further comprising a debris diversion assembly adapted to mechanically divert said debris particles away from a vicinity of said first articulating surface, towards said chamber.

69. The prosthetic device of claim 57, wherein a debris diversion assembly, including said first articulating surface and said sealing member, is adapted to mechanically divert said debris particles from a vicinity of at least one of said articulating surfaces, towards said chamber.

70. The prosthetic device of claim 63, wherein said first passageway is adapted to vary in width, responsive to a relative motion between said articulating surfaces.

71. The prosthetic device of claim 63, said articulating surfaces and said sealing face adapted wherein, as said first articulating surface rotates downward with respect to said second articulating surface, said sealing face is urged downward therewith, whereby an opening to said first passageway is widened.

72. The prosthetic device of claim 63, said articulating surfaces and said sealing face adapted wherein, as said first articulating surface rotates upward with respect to said second articulating surface, said sealing face is urged upward therewith, whereby an opening to said first passageway is narrowed.

73. The prosthetic device of claim 63, wherein said first passageway is adapted to deliver a fluid to said at least one chamber, said fluid laden with said debris particles, and wherein said chamber has a second passageway adapted to withdraw said fluid from said at least one chamber, said fluid being debris-depleted with respect to said fluid laden with said debris particles.

74. A prosthetic implant device designed to provide protection against debris-related dysfunction when implanted in a living body of a human or an animal, the prosthetic implant device comprising:
(a) a ball-and-socket prosthesis including at least:
(i) a ball component having a first articulating surface, and
(ii) a socket component having a second articulating surface,
wherein, in a working configuration, said socket component is adapted to at least partially receive said ball component, to form an articulating region disposed directly between said surfaces, and said first surface is adapted to move in a relative motion with respect to said second surface;
(b) a sealing arrangement, associated with said prosthesis, said arrangement including a sealing member having a sealing face disposed substantially opposite said first articulating surface, and abutting a portion thereof, to form a sealing region adapted to obstruct a discharge of debris particles disposed directly between said articulating surfaces, from said ball-and-socket prosthesis, via said sealing region, and
(c) at least one chamber, associated with said sealing arrangement, and adapted to receive and contain said debris particles,
wherein said ball-and-socket prosthesis is a hip prosthesis.

75. The prosthetic device of claim 74, said chamber disposed outside said articulating region.

76. A prosthetic implant device designed to provide protection against debris-related dysfunction when implanted in a living body of a human or an animal, the prosthetic implant device comprising:
(a) a ball-and-socket prosthesis including at least:
(i) a ball component having a first articulating surface, and
(ii) a socket component having a second articulating surface,
wherein, in a working configuration, said socket component is adapted to at least partially receive said ball component, to form an articulating region disposed directly between said surfaces, and said first surface is adapted to move in a relative motion with respect to said second surface;
(b) a sealing arrangement, associated with said prosthesis, said arrangement including a sealing member having a sealing face disposed substantially opposite said first articulating surface, and abutting a portion thereof, to form a sealing region adapted to obstruct a discharge of debris particles disposed directly between said articulating surfaces, from said ball-and-socket prosthesis, via said sealing region, and
(c) at least one chamber, associated with said sealing arrangement, and adapted to receive and contain said debris particles, and
(d) a debris diversion assembly adapted to mechanically divert said debris particles away from a vicinity of said first articulating surface, towards said chamber,
wherein said ball-and-socket prosthesis is a hip prosthesis.
